(19) United States
(12) Patent Application Publication
(10) Pub. No.: US 2022/0400996 A1
(43) Pub. Date: Dec. 22, 2022

(54) STRESS ANALYSIS APPARATUS, STRESS ANALYSIS METHOD, AND COMPUTER-READABLE RECORDING MEDIUM

(71) Applicant: NEC Corporation, Minato-ku, Tokyo (JP)

(72) Inventor: Tasuku KITADE, Tokyo (JP)

(73) Assignee: NEC Corporation, Minato-ku, Tokyo (JP)

(21) Appl. No.: 17/765,929

(22) PCT Filed: Oct. 8, 2019
(86) PCT No.: PCT/JP2019/039716

§ 371 Date: Apr. 1, 2022

(51) Int. Cl. A61B 5/16; A61B 5/00

(52) U.S. Cl. CPC A61B 5/165; A61B 5/7278; A61B 5/6801

(57) ABSTRACT

A stress analysis apparatus includes a stress level estimation unit for estimating a stress level of a user from biological information of the user, a stress state specification unit for specifying a time period during which the user is in a preset stress state, based on a time-series change of the estimated stress level, and a stress cause association unit for associating the specified time period with information regarding the user.

### TECHNICAL FIELD

The invention relates to a stress analysis apparatus and a stress analysis method for analyzing stress that impacts on a person, and further relates to a computer-readable recording medium that includes a program recorded thereon for realizing the apparatus and method.

### BACKGROUND ART

People usually experience stress due to various external stimuli. In particular, such stress that occurs at work is called occupational stress and occurs due to work and human relationship in a workplace. Occupational stress often leads to development of mental illness such as depression, and consequently causes a decrease in productivity, job departures, job absences, and the like. For this reason, it is necessary for companies to realize early detection of stress on employees.

For this reason, attempts have been conventionally made to quantify the level of stress on people. For example, Non-Patent Document 1 discloses a method for calculating a feature amount of stress that impacts on a subject. Specifically, in the method disclosed in Non-Patent Document 1, first, triaxial acceleration signals are obtained for a month as biological information from a wearable sensor attached to a subject, for example. Then, in the method disclosed in Non-Patent Document 1, at an arbitrary point during the month, a ratio of the obtained triaxial acceleration signals to acceleration signals obtained on a monthly basis is derived, and this ratio is taken as a feature amount indicating the stress level at that point.

In this manner, according to the method disclosed in Non-Patent Document 1, the state of the stress level in the target month can be specified from the amount of body movement of the subject in the target month. Non-Patent Document 2 discloses a method for calculating a feature amount of stress on a subject on a weekly basis, instead of a monthly basis, using a wearable sensor that is similar to the wearable sensor disclosed in Non-Patent Document 1.

### LIST OF RELATED ART DOCUMENTS

### Non-Patent Document

Non-Patent Document 1: Sano et al., “Recognizing academic performance, sleep quality, stress level, and mental health using personality traits, wearable sensors and mobile phones,” published in 2015 IEEE 12th International Conference on Wearable and Implantable Body Sensor Networks (BSN), 2015, pp. 1-6.
Non-Patent Document 2: Yoshiki Nakashima et al., “Improvement in Chronic Stress Level Recognition by Using Both Full-term and Short-term Measurements of Physiological Features”, The 32nd Annual Conference of the Japanese Society for Artificial Intelligence, 2018.

### SUMMARY OF INVENTION

### Problems to be Solved by the Invention

In this manner, according to the methods disclosed in Non-Patent Documents 1 and 2, since a change of the level of stress on a person can be quantified on a monthly or weekly basis, this can conceivably contribute to early detection of stress.

However, merely quantifying a change of the stress level is insufficient to reduce the stress, and the cause of the stress needs to be specified. In these methods, specification of the cause of the stress needs to be performed manually. For this reason, there is a problem in that it is difficult to fundamentally reduce stress with these methods.

In order to solve the above-described problem, an example object of the invention is to provide a stress analysis apparatus, a stress analysis method, and a computer-readable recording medium with which the state and cause of stress can be specified from biological information of a person.

### Means for Solving the Problems

In order to achieve the aforementioned example object, a stress analysis apparatus according to an example aspect of the invention is a stress analysis apparatus including

a stress level estimation unit configured to estimate a stress level of a user from biological information of the user,

a stress state specification unit configured to specify a time period during which the user is in a preset stress state, based on a time-series change of the estimated stress level, and

a stress cause association unit configured to associate the specified time period with information regarding the user.

Further, in order to achieve the aforementioned example object, a stress analysis method according to an example aspect of the invention is a stress analysis method including

(a) a step of estimating a stress level of a user from biological information of the user,

(b) a step of specifying a time period during which the user is in a preset stress state, based on a time-series change of the estimated stress level, and

(c) a step of associating the specified time period with information regarding the user.

Further, in order to achieve the aforementioned example object, a computer-readable recording medium according to an example aspect of the invention is a computer-readable recording medium that includes a program recorded thereon, the program further including instructions that cause a computer to carry out

(a) a step of estimating a stress level of a user from biological information of the user,

(b) a step of specifying a time period during which the user is in a prese stress state, based on a time-series change of the estimated stress level, and

(c) a step of associating the specified time period with information regarding the user.

### Advantageous Effects of the Invention

As described above, according to the invention, the state and cause of stress can be specified from biological information of a person.

### BRIEF DESCRIPTION OF THE DRAWINGS

FIG. 1 is a block diagram showing a schematic configuration of a stress analysis apparatus according to a first example embodiment.

FIG. 2 is a block diagram showing the configuration of the stress analysis apparatus according to the first example embodiment in more detail.

FIG. 3 is a flowchart showing the operations of the stress analysis apparatus according to the first example embodiment.

FIG. 4 is a diagram showing an example of information displayed on a screen of a terminal device in the first example embodiment.

FIG. 5 is a block diagram showing a configuration of a stress analysis apparatus according to a second example embodiment.

FIG. 6 is a flowchart showing the operations of the stress analysis apparatus according to the second example embodiment.

FIG. 7 is a block diagram showing one example of a computer that realizes the stress analysis apparatus according to the first and second example embodiments of the invention.

### EXAMPLE EMBODIMENT

### First Example Embodiment

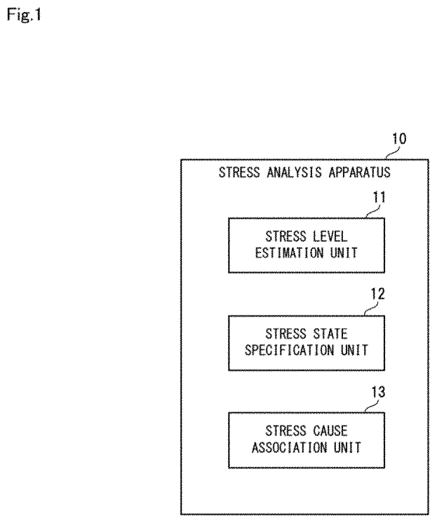

The following describes a stress analysis apparatus, a stress analysis method, and a program according to a first example embodiment with reference to FIG. 1 to FIG. 4.

### Apparatus Configuration

First, a schematic configuration of the stress analysis apparatus according to the first example embodiment will be described using FIG. 1. FIG. 1 is a block diagram showing a schematic configuration of a stress analysis apparatus according to the first example embodiment.

A stress analysis apparatus 10 according to the first example embodiment shown in FIG. 1 is an apparatus for analyzing stress that impacts on a user. As shown in FIG. 1, the stress analysis apparatus 10 is provided with a stress level estimation unit 11, a stress state specification unit 12, and a stress cause association unit 13.

The stress level estimation unit 11 estimates the stress level of the user from biological information of the user. The stress state specification unit 12 specifies a time period during which the user is in a preset stress state, based on a time-series change of the stress level estimated by the stress level estimation unit 11. The stress cause association unit 13 associates the time period specified by the stress state specification unit 12 with information regarding the user.

Here, examples of a preset stress state include a high-stress state that may damage the user's health and a low-stress state in which the user feels no stress. Also, examples of the information regarding the user include information regarding events which the user attends (event name, type of event, attendees, start time, end time, event duration, place, etc.), information regarding the environment of the user (weather, room temperature, outside temperature, humidity, etc.), and information regarding people involved with the user (supervisors, colleagues, friends, family, etc.). In the present specification, “user” includes not only a person from whom biological information is obtained, but also a person who is in charge of supervising the person, such as a manager of a company or a parent.

In the first example embodiment, with this configuration, the time period during which the user is in the high-stress state is specified, and the specified time period and information regarding the user at that time are associated with each other. That is, according to the first example embodiment, the state and cause of stress can be specified from biological information of a person.

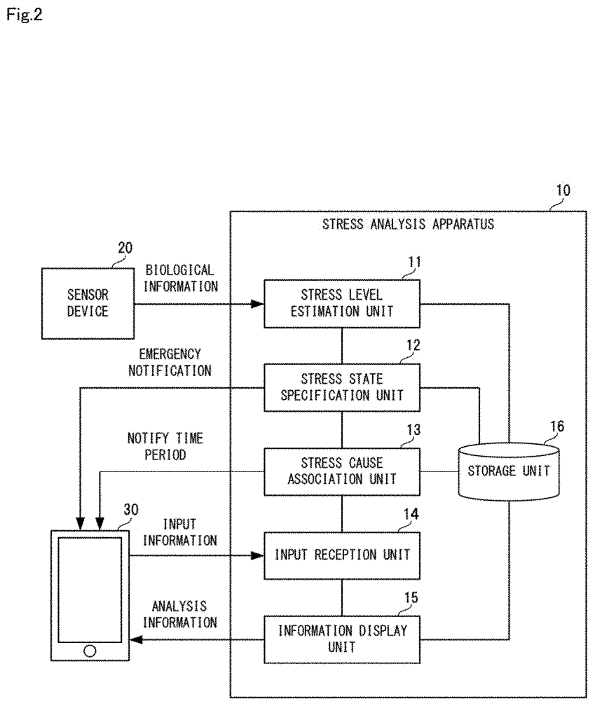

Next, the configuration of the stress analysis apparatus 10 according to the first example embodiment will be described more specifically using FIG. 2. FIG. 2 is a block diagram showing the configuration of the stress analysis apparatus according to the first example embodiment in more detail.

As shown in FIG. 2, in the first example embodiment, the stress analysis apparatus 10 is connected to a sensor device 20 for obtaining biological information of the user, and to a terminal device 30 of the user. In the first example embodiment, the stress analysis apparatus 10 can also be constructed inside the terminal device 30 by a program according to the first example embodiment, which will be described later, being installed in a computer of the terminal device 30 and executed. Examples of the terminal device 30 include a smartphone, a tablet-type terminal, and a laptop PC (Personal Computer).

The sensor device 20 is provided with a sensor that can detect biological information, and outputs the detected biological information. Examples of the sensor device 20 include a pulse rate meter for detecting heart rate, a skin electrometer for detecting the amount of skin electrical activity, a sweat meter for detecting the amount of sweating, an acceleration meter for detecting the acceleration of movement of a person. The sensor device 20 may also be a camera. In this case, the stress level estimation unit 11 estimates the stress level from an image of a human body. Also, communication between the sensor device 20 and the stress analysis apparatus 10 may be performed wirelessly or by cable.

As shown in FIG. 2, in the first example embodiment, the stress analysis apparatus 10 is provided with an input reception unit 14, an information display unit 15, and a storage unit 16, in addition to the stress level estimation unit 11, the stress state specification unit 12, and the stress cause association unit 13.

In the first example embodiment, the stress level estimation unit 11 obtains the biological information output from the sensor device 20, and estimates the stress level of the user from the obtained biological information. Specifically, for example, if the sensor device 20 is an acceleration meter, the stress level estimation unit 11 obtains acceleration indicating the movement of the body of the user as biological information. Then, the stress level estimation unit 11 estimates the stress level from the obtained acceleration according to the methods disclosed in the above-described Non-Patent Document 1 or 2.

If the sensor device 20 is a pulse rate meter, the stress level estimation unit 11 obtains the heart rate of the user as the biological information. If the sensor device 20 is a skin electrometer, the stress level estimation unit 11 obtains the amount of skin electrical activity of the user as the biological information, and if the sensor device 20 is a sweat meter, the stress level estimation unit 11 obtains the amount of sweating as the biological information. Then, the stress level estimation unit 11 estimates the stress level using a method corresponding to the type of the obtained biological information.

Also, in the first example embodiment, an existing method, and further, a method which will be developed in the future can be used as a method for estimating the stress level from the acceleration, heart rate, amount of skin electrical activity, and amount of sweating. Further, the stress level estimation unit 11 stores the estimated stress level in the storage unit 16 in a chronological order, specifically, in a state where information regarding the time at which the biological information was output from the sensor device 20 is appended thereto.

Also, in the first example embodiment, the sensor device 20 may also be provided with a plurality of types of sensors and output a plurality of types of biological information corresponding to the sensors. In this case, the stress level estimation unit 11 can also obtain the plurality of types of biological information and estimate the stress level using the plurality of types of biological information.

In the first example embodiment, first, the stress state specification unit 12 specifies a time-series change of the stress level from the stress levels stored in the storage unit 16. Next, the stress state specification unit 12 specifies a time period during which the user is in a predetermined stress state, based on the time-series change of this stress level, and stores information indicating the specified time period as time period information in the storage unit 16.

A predetermined stress state is set in advance by the user of the stress analysis apparatus, for example. Examples of such a stress state include a state in which the user may have mental and physical problems, that is, the high-stress state.

If a predetermined state is the high-stress state, the stress state specification unit 12 determines whether the user is in the high-stress state according to any of criteria (a) to (d) shown below, and specifies the time period, for example. Note that, hereinafter, X denotes the value of the stress level, and θh denotes a reference value (threshold) of the stress level. The reference value (threshold value) θh is also appropriately set by the user of the stress analysis apparatus 10 or the like.

(a) If X>θh occurs n or more times within a set time period (e.g., one hour), it is determined to be the high-stress state, and the time period from the time point when X>θh was first satisfied to the time point when X>θ was last satisfied is taken as a high-stress state time period.

(b) If the time period in which X>θh is maintained within a set time period (e.g., one day) exceeds T time, it is determined to be the high-stress state, and the time period from the time point when X>θh is satisfied for the first time to the time point when X>θh is no longer satisfied is taken as the high-stress state time period.

(c) When a fluctuation range ΔX of X per unit time reaches a certain value or more, it is determined to be the high-stress state, and the time period from the time point when ΔX reaches a certain value or more for the first time to the time point when ΔX becomes less than the certain value is taken as the high-stress state time period.

(d) It is assumed that the fluctuation range ΔX of X per unit time for a past Tp time from the set time satisfies a certain requirement, and from that fact it can be expected that X will exceed θh before Tf time elapses from the set time. In this case, it is determined to be the high-stress state, and the time period from the time point when the past Tp time from the set time to the time point when X becomes θh or less after exceeding θh is taken as the high-stress state time period.

Further, if the specified stress state is an extremely high-stress state or the like, the stress state specification unit 12 can also notify the terminal device 30 of that fact. In this manner, the user can recognize that his or her own stress state is extremely high, and a situation where the health of the user is damaged can be prevented. Note that the timing of the notification is appropriately set by the user of the stress analysis apparatus 10 or the like.

In the first example embodiment, first, the stress cause association unit 13 obtains time period information that specifies the time period during which the user is in a predetermined stress state, from the storage unit 16, and notifies the terminal 30 of the user of the obtained time period information. In this manner, the terminal device 30 of the user displays, on the screen, the time period during which the user is in a predetermined stress state, such as the high-stress state, for example.

In this case, the user confirms the time period displayed on the terminal device 30, and inputs the information regarding the user in that time period to the terminal device 30. In this manner, the terminal device 30 transmits the input information to the stress analysis apparatus 10 as input information. Examples of the information regarding the user include, as described above, information about events which the user attends, information regarding the environment of the user, and information regarding people involved with the user, the level of stress the user feel, the content of a questionnaire taken by the user and the answers thereof. Specifically, if the user has attended a meeting of the company in the time period displayed on the terminal device 30, the user inputs the title of the meeting, type of meeting, names of attendees, start time, end time, duration of meeting, place, and the like.

Once the input information is transmitted to the stress analysis apparatus 10, the input reception unit 14 receives the input information, and inputs the received information to the stress cause association unit 13. The stress cause association unit 13 associates this input information with the time period information, and stores these two types of information in association with each other in the storage unit 16.

The information display unit 15 displays, on the screen, the time period specified by the stress state specification unit 12 and the information regarding the user in association with each other. Specifically, the information display unit 15 obtains, from the storage unit 16, the time period information and the input information associated with each other, and transmits the obtained information to the terminal device 30 and displays the information on the screen.

In this manner, the user can understand when his or her stress level is high at a glance from the information displayed on the screen of the terminal device 30 of the user.

### Apparatus Operations

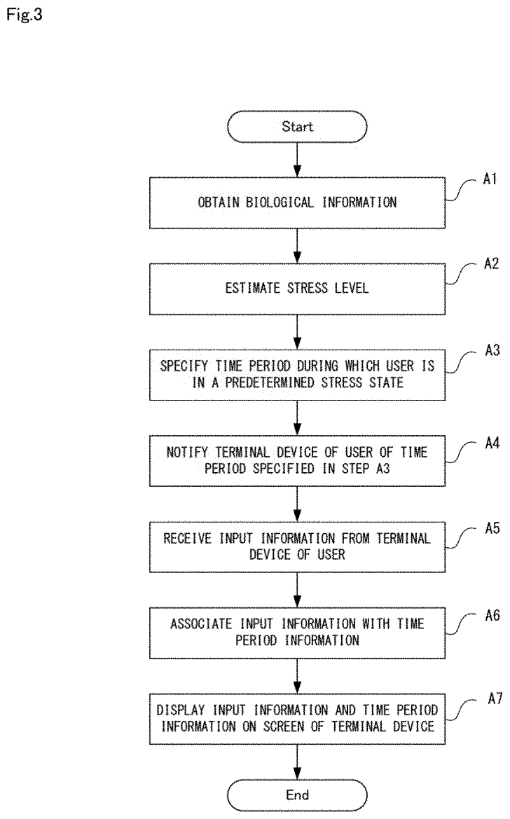

Next, the operations of the stress analysis apparatus 10 according to the first example embodiment will be described using FIG. 3. FIG. 3 is a flowchart showing the operations of the stress analysis apparatus according to the first example embodiment. In the description below, FIGS. 1 and 2 are referred to as appropriate. In the first example embodiment, the stress analysis method is implemented by operating the stress analysis apparatus 10. Therefore, the following description of the operations of the stress analysis apparatus 10 will be given in place of a description of the stress analysis method of the example embodiment.

As shown in FIG. 3, first, the stress level estimation unit 11 obtains the biological information that is output from the sensor device 20 (step A1).

Next, the stress level estimation unit 11 estimates the stress level of the user from the biological information obtained in step A1 (step A2). Also, after executing step A2, the stress level estimation unit 11 stores the stress level estimated in step A2 in the storage unit 16 in time series.

Next, the stress state specification unit 12 specifies the time-series change of the stress level from the stress level estimated in step A2, and specifies the time period during which the user is in a predetermined stress state (high-stress state) from the specified time-series change of the stress level (step A3). Also, the stress state specification unit 12 stores the specified time period in the storage unit 16 as the time period information.

Next, the stress cause association unit 13 notifies the time period specified in step A3 of the terminal device 30 of the user (step A4). Specifically, the stress cause association unit 13 obtains the time period information from the storage unit 16, and notifies the obtained time period information of the terminal device 30 of the user.

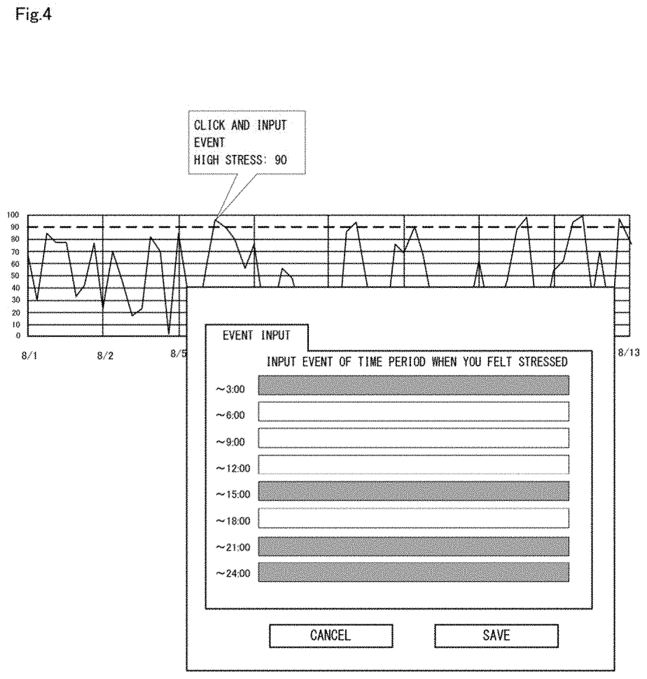

After executing step A4, as shown in FIG. 4, the terminal device 30 of the user displays the time period during which the user is in a predetermined stress state on the screen. Then, as shown in FIG. 4, the user confirms the time period displayed on the terminal device 30, and inputs the information regarding the user in the time period to the terminal device 30. In this manner, the terminal device 30 transmits the input information to the stress analysis apparatus 10 as input information. FIG. 4 is a diagram showing an example of information displayed on the screen of the terminal device in the first example embodiment.

Next, upon receiving the input information, the input reception unit 14 receives the input information and inputs the received input information to the stress cause association unit 13 (step A5). Next, the stress cause association unit 13 associates the input information with the time period information, and stores these two types of information in association with each other in the storage unit 16 (step A6).

Next, the information display unit 15 obtains, from the storage unit 16, the input information and the time period information that are associated with each other in step A6, transmits the information to the terminal device 30, and displays the information on the screen of the terminal device 30 (step A7).

### Effects of First Example Embodiment

As described above, in the first example embodiment, the time period during which the user is in a specified state such as the high-stress state is specified, and the specified time period and the information regarding the user at that time are associated with each other, and such association is displayed on the screen. In this manner, the user can understand when his or her stress level is high at a glance from the information displayed on the screen of the terminal device 30 of the user.

### Variations

Although the above example describes the case in which a predetermined stress state is a high-stress state, in the first example embodiment, a predetermined stress state may also be a low-stress state in which the user feels no psychological burden. In this case, the stress state specification unit 12 specifies the time period in which the user is in the low-stress state. Further, if the stress state specification unit 12 can specify the time period in which the user is in the low-stress state, the fact that the high-stress state is mitigated can also be detected. Further, in this case, the stress cause association unit 13 associates the time period in which the user is in the low-stress period with the information regarding the user.

The stress state specification unit 12 determines whether the user is in the low-stress state according to any one of the following criteria (e) to (h), for example, and specifies the time period. Note that, in the following description, X denotes the value of the stress level, and θI denotes the reference value of the stress level (threshold value). The reference value (threshold value) θI is appropriately set by the user of the stress analysis apparatus 10 or the like.

(e) When the state shifts from a state where X>θh to a state where X≤θh, it is determined to be the low-stress state, and the time period from the time point when X≤θn is satisfied to the time point when X>θh is next satisfied is taken as a low-stress state time period.

(f) When a fluctuation range ΔX of X per unit time is less than a certain value, it is determined to be the low-stress state, and the time period from the time point when ΔX first becomes less than a certain value to the time point when ΔX reaches the certain value or more is taken as a low-stress state time period.

(g) If X<θI occurs n or more times within a set time period (e.g., one hour), it is determined to be the low-stress state, and the time period from the time point when X<θI was first satisfied to the time point when X<θI was last satisfied is taken as a low-stress state time period.

(h) When X<θI is satisfied, it is determined to be a low-stress state, and the time period from the time point when X<θI is first satisfied to the time point when X≥θI is satisfied is taken as a low-stress state time period.

Further, if the stress state specification unit 12 determines that the user is in the low-stress state according to the above criteria (e) to (h) in a case where the stress state specification unit 12 has determined that the user is in the high-stress state according to the above criteria (a) to (d), the stress state specification unit 12 may also notify the terminal device 30 of this fact. In this manner, the user can recognize that the stress state of the user has been mitigated, and feel at ease. Note that the timing of the notification is appropriately set by the user of the stress analysis apparatus 10 or the like.

Further, the stress state specification unit 12 can also specify the time period in which the user is in the high-stress state and the time period in which the user is in the low-stress state. In this case, the stress cause association unit 13 associates the time period in which the user is in the low-stress state with the information regarding the user, in addition to associating the time period in which the user is in the high-stress state with the information regarding the user.

### Program

A program according to the first example embodiment need only be a program that causes a computer to execute steps A1 to A7 shown in FIG. 3. The stress analysis apparatus 10 and the stress analysis method according to the first example embodiment can be realized by this program being installed on a computer and executed. In this case, a processor of the computer functions and performs processing as the stress level estimation unit 11, the stress state specification unit 12, the stress cause association unit 13, the input reception unit 14, and the information display unit 15. Further, the storage unit 16 can be realized by storing a data file constituting the above in a storage device such as hard disk provided in the computer. Further, the computer may be a general-purpose computer, or a computer mounted in the terminal device 30 of the user.

Also, the program in the first example embodiment may be executed by a computer system that is constituted by a plurality of computers. In this case, for example, each computer may function as one of the stress level estimation unit 11, the stress state specification unit 12, the stress cause association unit 13, the input reception unit 14, and the information display unit 15. Further, the storage unit 16 may be structured on a computer separate from the computer that executes the program according to the first example embodiment.

### Second Example Embodiment

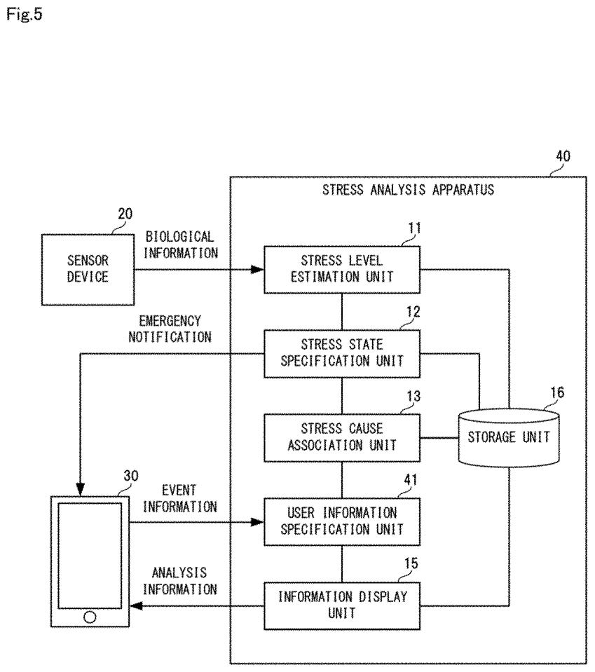

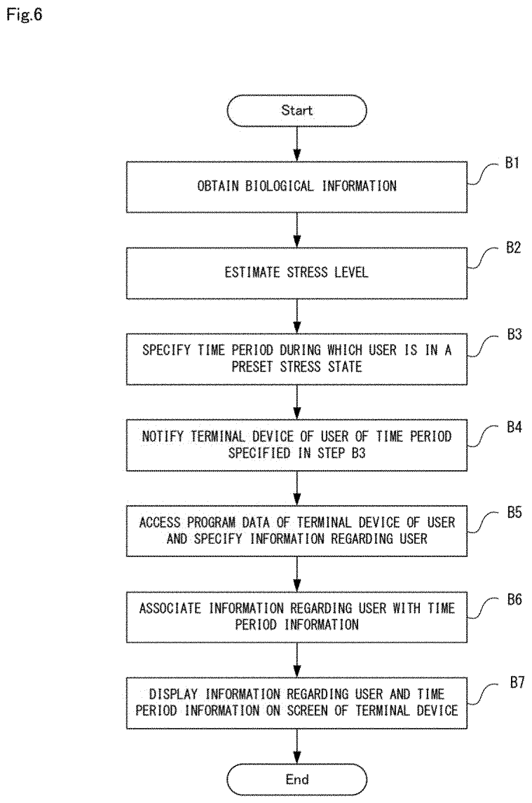

Next, a stress analysis apparatus, a stress analysis method, and a program according to a second example embodiment will be described with reference to FIGS. 5 and 6.

### Apparatus Configuration

First, a configuration of the stress analysis apparatus according to a second example embodiment will be described using FIG. 5. FIG. 5 is a block diagram showing the configuration of the stress analysis apparatus according to the second example embodiment.

As shown in FIG. 5, similarly to the stress analysis apparatus 10 according to the first example embodiment shown in FIG. 2, a stress analysis apparatus 40 according to the second example embodiment is provided with the stress level estimation unit 11, the stress state specification unit 12, the stress cause association unit 13, the information display unit 15, and the storage unit 16. The stress analysis apparatus 40 according to the second example embodiment is also connected to the sensor device 20 and the terminal device 30 of the user.

Note that the stress analysis apparatus 40 according to the second example embodiment is provided with a user information specification unit 41 instead of the input reception unit 14 shown in FIG. 2, and is different from the stress analysis apparatus 10 according to the first example embodiment in this regard. Hereinafter, differences from the first example embodiment will be mainly described.

The user information specification unit 41 accesses data of a program installed in the terminal device 30 of the user. Then, the user information specification unit 41 specifies information regarding the user in the time period specified by the stress state specification unit 12 by accessing the data. In the second example embodiment, the stress cause association unit 13 associates the information regarding the user which is specified by the user information specification unit 41 with the time period specified by the stress state specification unit 12.

Specifically, for example, it is assumed that a schedule management program is installed in the terminal device 30 of the user. In this case, the user information specification unit 41 obtains the data that specifies the schedule of the user from the schedule management program. Then, the user information specification unit 41 specifies information regarding an event which the user attended in the period that overlaps the time period specified by the stress state specification unit 12 from the obtained data. If the event is a work meeting, examples of information regarding the event include, the title of the meeting, the type of meeting, the names of attendees, the start time, the end time, the duration of the meeting, and the place.

If a program that functions as a communication tool is installed in the terminal device 30 of the user, the user information specification unit 41 obtains, as information regarding the user, the name of the person contacted, the location, the contents of the topic, the conversation history, and the like, from the communication history.

Further, the user information specification unit 41 can also obtain, as information regarding the user, image data, activity history (acceleration information, movement distance, amount of activity, number of steps, GPS (Global Positioning System) information, etc.) stored in the terminal device 30. The user information specification unit 41 can also access an external database over the Internet to obtain meteorological information (weather, outside temperature, humidity, etc.) in a period that coincides with the time period specified by the stress state specification unit 12, as information regarding the user.

### Apparatus Operations

Next, the operations of the stress analysis apparatus 40 according to the second example embodiment will be described using FIG. 6. FIG. 6 is a flowchart showing the operations of the stress analysis apparatus according to the second example embodiment. In the description below, FIG. 5 is referred to as appropriate. In the second example embodiment, the stress analysis method is implemented by operating the stress analysis apparatus 40. Therefore, the following description of the operations of the stress analysis apparatus 40 will be given in place of a description of the stress analysis method in the example embodiment.

As shown in FIG. 6, first, the stress level estimation unit 11 obtains the biological information that is output from the sensor device 20 (step B1). Next, the stress level estimation unit 11 estimates the stress level of the user from the biological information obtained in step A1 (step B2). Steps B1 and B2 are similar to steps A1 and A2 shown in FIG. 3.

Next, the stress state specification unit 12 specifies a time-series change of the stress level from the stress level estimated in step B2, and estimates the time period in which the user is in a predetermined stress state (e.g., high-stress state, low-stress state, or both) from the specified time-series change of the stress level (step B3). Also, the stress state specification unit 12 stores information indicating the specified time period in the storage unit 16 as the time period information.

Next, the stress cause association unit 13 notifies the terminal device 30 of the user of the time period specified in step B3 (step B4). Steps B3 and B4 are similar to steps A3 and A4 shown in FIG. 3.

After executing step B4, the user information specification unit 41 accesses data of a program installed in the terminal device 30 of the user and specifies the information regarding the user in the time period specified in step B3 (step B5). Also, the user information specification unit 41 inputs the specified information regarding the user to the stress cause association unit 13.

Next, the stress cause association unit 13 associates the information regarding the user specified in step B5 with the time period information and stores the associated two types of information in the storage unit 16 (step B6).

Next, the information display unit 15 obtains, from the storage unit 16, the information regarding the user and the time period information that are associated with each other in step A6, and transmits the information to the terminal device 30 to be displayed on the screen of the terminal device 30 (step B7).

### Effects of Second Example Embodiment

As described above, in the second example embodiment, when the time period in which the user is in a specific state such as the high-stress state is specified, information regarding the user related to the time period will be automatically obtained from the terminal device 30. Unlike the first example embodiment, the user does not need to input the information. For this reason, according to the second example embodiment, the user can grasp when his or her stress level is high at a glance, in a simpler manner than the first example embodiment.

### Program

A program according to the second example embodiment need only be a program that causes a computer to execute steps B1 to B7 shown in FIG. 6. The stress analysis apparatus 40 and the stress analysis method according to the second example embodiment can be realized by this program being installed on a computer and executed. In this case, the processor of the computer functions and executes processing as the stress level estimation unit 11, the stress state specification unit 12, the stress cause association unit 13, the information display unit 15, and the user information specification unit 41. Further, the storage unit 16 can be realized by storing a data file constituting the above in a storage device such as hard disk provided in the computer. Further, the computer may be a general-purpose computer, or a computer mounted in the terminal device 30 of the user.

Also, the program of the second example embodiment may also be executed by a computer system constituted by a plurality of computers. In this case, for example, each computer may function as one of the stress level estimation unit 11, the stress state specification unit 12, the stress cause association unit 13, the information display unit 15, and the user information specification unit 41. Further, the storage unit 16 may also be constructed in another computer separate from the computer that executes the program according to the second example embodiment.

### Physical Configuration

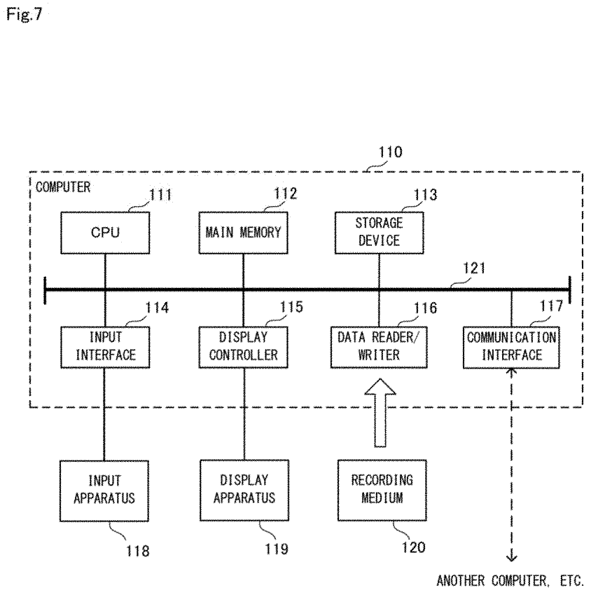

Here, a computer that realizes the stress analysis apparatus by executing a program of the first and second example embodiments will be described, using FIG. 7. FIG. 7 is a block diagram showing an example of the computer that realizes the stress analysis apparatus according to the first and second example embodiments of the invention.

As shown in FIG. 7, a computer 110 includes a CPU (Central Processing Unit) 111, a main memory 112, a storage device 113, an input interface 114, a display controller 115, a data reader/writer 116, and a communication interface 117. These components are connected in such a manner that they can perform data communication with one another via a bus 121. The computer 110 may include a GPU (Graphics Processing Unit) or an FPGA (Field-Programmable Gate Array), in addition to the CPU 111 or instead of the CPU 111.

The CPU 111 carries out various computational operations by deploying the program (codes) in the present example embodiment stored in the storage device 113 to the main memory 112, and executing the deployed programs in a predetermined order. The main memory 112 is typically a volatile storage device, such as a DRAM (Dynamic Random Access Memory). Also, the program in the present example embodiment is provided in a state where it is stored in a computer-readable recording medium 120. Note that the program in the present example embodiment may also be distributed over the Internet connected via the communication interface 117.

Furthermore, specific examples of the storage device 113 include a hard disk drive, and also a semiconductor storage device, such as a flash memory. The input interface 114 mediates data transmission between the CPU 111 and an input apparatus 118, such as a keyboard and a mouse. The display controller 115 is connected to a display apparatus 119, and controls display on the display apparatus 119.

The data reader/writer 116 mediates data transmission between the CPU 111 and the recording medium 120, and executes readout of the program from the recording medium 120, as well as writing of the result of processing in the computer 110 to the recording medium 120. The communication interface 117 mediates data transmission between the CPU 111 and another computer.

Also, specific examples of the recording medium 120 include: a general-purpose semiconductor storage device, such as CF (Compact Flash®) and SD (Secure Digital); a magnetic recording medium, such as Flexible Disk; and an optical recording medium, such as CD-ROM (Compact Disk Read Only Memory).

Note that the stress analysis apparatus in the first and second example embodiments can also be realized using items of hardware corresponding to respective components, rather than using the computer with the program installed therein. Furthermore, a part of the stress analysis apparatus may be realized by the program, and the remaining part of the stress analysis apparatus may be realized by hardware.

A part or all of the aforementioned example embodiment can be described as, but is not limited to, the following (Supplementary Note 1) to (Supplementary Note 15).

(Supplementary Note 1)

A stress analysis apparatus including:

a stress level estimation unit configured to estimate a stress level of a user from biological information of the user;

a stress state specification unit configured to specify a time period during which the user is in a preset stress state, based on a time-series change of the estimated stress level; and

a stress cause association unit configured to associate the specified time period with information regarding the user.

(Supplementary Note 2)

The stress analysis apparatus according to supplementary note 1, further including:

a user information specification unit configured to access data of a program installed in a terminal device of the user and specifying information regarding the user in the specified time period,

wherein the stress cause association unit associates the information regarding the user that is specified by the user information specification unit with the specified time period.

(Supplementary Note 3)

The stress analysis apparatus according to supplementary note 2,

wherein the user information specification unit accesses a schedule management program that is installed in the terminal device of the user, and specifies, as the information regarding the user, information about an event which the user attended in a period that coincides with the specified time period.

(Supplementary Note 4)

The stress analysis apparatus according to any one of supplementary notes 1 to 3, further including:

an information display unit configured to display the specified time period and the information regarding the user in association with each other on a screen.

(Supplementary Note 5)

The stress analysis apparatus according to any one of supplementary notes 1 to 4,

wherein the stress state specification unit specifies a first time period during which the user is in a first stress state and a second time period during which the user is in a second stress state in which the user is less stressed than the first stress state.

(Supplementary Note 6)

A stress analysis method including:

(a) a step of estimating a stress level of a user from biological information of the user;

(b) a step of specifying a time period during which the user is in a preset stress state, based on a time-series change of the estimated stress level; and

(c) a step of associating the specified time period with information regarding the user.

(Supplementary Note 7)

The stress analysis method according to supplementary note 7, further including:

(d) a step of accessing data of a program installed in a terminal device of the user and specifying information regarding the user in the specified time period,

wherein, in the (c) step, the information regarding the user that is specified in the (d) step is associated with the specified time period.

(Supplementary Note 8)

The stress analysis method according to supplementary note 7,

in the (d) step, a schedule management program installed in the terminal device of the user is accessed, and information about an event which the user attended in a period that coincides with the specified time period is specified as the information regarding the user.

(Supplementary Note 9)

The stress analysis method according to any one of supplementary notes 6 to 8, further including:

(e) displaying the specified time period and the information regarding the user in association with each other on a screen.

(Supplementary Note 10)

The stress analysis method according to any one of supplementary notes 6 to 9,

in the (c) step, a first time period during which the user is in a first stress state and a second time period during which the user is in a second stress state in which the user is less stressed than the first stress state are specified.

(Supplementary Note 11)

A computer-readable recording medium that includes a program recorded thereon, the program further including instructions that cause a computer to carry out:

(a) a step of estimating a stress level of a user from biological information of the user;

(b) a step of specifying a time period during which the user is in a prese stress state, based on a time-series change of the estimated stress level; and

(c) a step of associating the specified time period with information regarding the user.

(Supplementary Note 12)

The computer-readable recording medium according to supplementary note 11, the program further including instructions that cause the computer to carry out:

(d) a step of accessing data of a program installed in a terminal device of the user, and specifying information regarding the user in the specified time period,

wherein, in the (c) step, the information regarding the user that is specified in the (d) step is associated with the specified time period.

(Supplementary Note 13)

The computer-readable recording medium according to supplementary note 12,

wherein, in the (d) step, a schedule management program installed in the terminal device of the user is accessed, and information about an event which the user attended in a period that coincides with the specified time period is specified as the information regarding the user.

(Supplementary Note 14)

The computer-readable recording medium according to any one of supplementary notes 11 to 13, the program further including instructions that cause the computer to carry out:

(e) displaying the specified time period and the information regarding the user in association with each other on a screen.

(Supplementary Note 15)

The computer-readable recording medium according to any one of supplementary notes 11 to 14,

wherein, in the (c) step, a first time period during which the user is in a first stress state and a second time period during which the user is in a second stress state in which the user is less stressed than the first stress state are specified.

Although the invention of the present application has been described above with reference to the example embodiments, the invention is not limited to the foregoing example embodiments. Various variations apparent to those skilled in the art can be made to the configurations and details of the invention of the present application within the scope of the invention.

### INDUSTRIAL APPLICABILITY

As described above, according to the invention, it is possible to specify the state and cause of stress from biological information of a person. The invention can be used in various fields in which analysis of stress on people is required.

### REFERENCE SIGNS LIST

10 Stress analysis apparatus (first example embodiment)
11 Stress level estimation unit
12 Stress state specification unit
13 Stress cause association unit
14 Input reception unit
15 Information display unit
16 Storage unit
20 Sensor device
30 Terminal device
40 Stress analysis apparatus (second example embodiment)
41 User information specification unit
110 Computer
111 CPU
112 Main memory
113 Storage device
114 Input interface
115 Display controller
116 Data reader/writer
117 Communication interface
118 Input device
119 Display device
120 Storage medium
121 Bus

What is claimed is:

1. A stress analysis apparatus comprising:
at least one memory storing instructions; and
at least one processor configured to execute the instructions to:
estimate a stress level of a user from biological information of the user;
specify a time period during which the user is in a preset stress state, based on a time-series change of the estimated stress level; and
associate the specified time period with information regarding the user.

2. The stress analysis apparatus according to claim 1, further at least one processor configured to execute the instructions to:
access data of a program installed in a terminal device of the user and specifying information regarding the user in the specified time period,
associate the information regarding the user that is specified by the user information specification means with the specified time period.

3. The stress analysis apparatus according to claim 2,
further at least one processor configured to execute the instructions to:
access a schedule management program that is installed in the terminal device of the user, and specifies, as the information regarding the user, information about an event which the user attended in a period that coincides with the specified time period.

4. The stress analysis apparatus according to claim 1, further at least one processor configured to execute the instructions to:
display the specified time period and the information regarding the user in association with each other on a screen.

5. The stress analysis apparatus according to claim 1,
further at least one processor configured to execute the instructions to:
specify a first time period during which the user is in a first stress state and a second time period during which the user is in a second stress state in which the user is less stressed than the first stress state.

6. A stress analysis method comprising:
estimating a stress level of a user from biological information of the user;
specifying a time period during which the user is in a preset stress state, based on a time-series change of the estimated stress level; and
associating the specified time period with information regarding the user.

7. The stress analysis method according to claim 6, further comprising:
accessing data of a program installed in a terminal device of the user and specifying information regarding the user in the specified time period,
wherein, in the associating, the information regarding the user that is specified in the accessing is associated with the specified time period.

8. The stress analysis method according to claim 7,
in the accessing, a schedule management program installed in the terminal device of the user is accessed, and information about an event which the user attended in a period that coincides with the specified time period is specified as the information regarding the user.

9. The stress analysis method according to claim 6, further comprising:
displaying the specified time period and the information regarding the user in association with each other on a screen.

10. The stress analysis method according to claim 6,
in the associating, a first time period during which the user is in a first stress state and a second time period during which the user is in a second stress state in which the user is less stressed than the first stress state are specified.

11. A non-transitory computer-readable recording medium that includes a program recorded thereon, the program further including instructions that cause a computer to carry out:
estimating a stress level of a user from biological information of the user;
specifying a time period during which the user is in a prese stress state, based on a time-series change of the estimated stress level; and
associating the specified time period with information regarding the user.

12. The non-transitory computer-readable recording medium according to claim 11, the program further including instructions that cause the computer to carry out:
accessing data of a program installed in a terminal device of the user, and specifying information regarding the user in the specified time period,
wherein, in the associating, the information regarding the user that is specified in the accessing is associated with the specified time period.

13. The non-transitory computer-readable recording medium according to claim 12,
wherein, in the accessing, a schedule management program installed in the terminal device of the user is accessed, and information about an event which the user attended in a period that coincides with the specified time period is specified as the information regarding the user.

14. The non-transitory computer-readable recording medium according to claim 11, the program further including instructions that cause the computer to carry out:
displaying the specified time period and the information regarding the user in association with each other on a screen.

15. The non-transitory computer-readable recording medium according to claim 14,
wherein, in the associating, a first time period during which the user is in a first stress state and a second time period during which the user is in a second stress state in which the user is less stressed than the first stress state are specified.
